(19) United States
(12) Patent Application Publication
(10) Pub. No.: US 2025/0041220 A1
(43) Pub. Date: Feb. 6, 2025

(54) METHODS AND SYSTEMS FOR POINT-OF-CARE SYNTHESIS AND ADMINISTRATION OF PARTICLE-BASED THERAPEUTICS

(71) Applicant: Verily Life Sciences LLC, South San Francisco, CA (US)

(72) Inventors: Mark Audeh, Brookline, MA (US); Eric Paoli, Los Altos, CA (US); Jasper Lodewyk Steyn, San Bruno, CA (US); Jaeden Hutchison, Oakland, CA (US)

(73) Assignee: Verily Life Sciences LLC, South San Francisco, CA (US)

(21) Appl. No.: 18/706,889

(22) PCT Filed: Aug. 18, 2022
(86) PCT No.: PCT/US2022/075109

§ 371 Date: May 2, 2024

Related U.S. Application Data
(60) Provisional application No. 63/263,621, filed on Nov. 5, 2021.

(51) Int. Cl. A61K 9/127; A61K 31/7088; A61M 5/32; B01D 61/24; B01F 23/40; B01F 23/45; B01F 23/80; B01F 33/301; B01F 35/71; B01F 101/22

(52) U.S. Cl. CPC A61K 9/127; A61K 31/7088; A61M 5/32; B01D 61/243; B01F 23/405; B01F 23/45; B01F 23/808; B01F 33/3017; B01F 35/712; B01F 35/7174; B01F 35/71805; B01F 2101/22

(57) ABSTRACT

The present disclosure is related to an apparatus and system for synthesizing and administering particle-based therapeutics at the point-of-care. The apparatus includes a first chamber for receiving a first solution including lipids in a first solvent, a second chamber for receiving a second solution including nucleic acids in a second solvent, a mixing channel in communication with the first chamber and the second chamber, and a delivery system in communication with the to the mixing channel. The first solution in the first chamber and the second solution in the second chamber can be introduced into the mixing channel. The particle-based delivery system form in the mixing channel and the nucleic acids adhere to the particles. The formulated particle-based therapeutics are passed through a delivery system to a subject for administration.

### CROSS-REFERENCE TO RELATED APPLICATION

This application claims the benefit of and priority to U.S. Provisional Application No. 63/263,621, filed on Nov. 5, 2021, the disclosure of which is hereby incorporated by reference in its entirety.

### BACKGROUND OF THE INVENTION

Nucleic acid administration based on plasmid DNA, viral vectors or messenger RNA (mRNA) has been evaluated for several clinical applications, including cancer, allergy and gene replacement therapies, and has proven to be an effective delivery system for vaccines against infectious diseases. There has been considerable focus on modified mRNA vaccines during the last decade, as they are safe, scalable, and offer precision in antigen design. For example, modified mRNA vaccines circumvent the problem of pre-existing immunity associated with viral vectors. mRNA vaccines may be especially valuable for emerging infections such as pandemic influenza. With the recent success of mRNA vaccines (e.g., Pfizer and Moderna's severe acute respiratory syndrome coronavirus 2 (SARS-CoV-2) vaccine), as well as the growing fields of gene replacement therapies, gene silencing therapies, and gene editing applications, there is a need for intact in vivo delivery of nucleic acids (e.g., RNA and/or DNA) into intact cells.

Currently, one method for delivering nucleic acids or small molecule therapeutics is the use of nanoparticles (e.g., lipid nanoparticles) or microparticles as a carrier for the nucleic acid or small molecule payload. For example, nanoparticles are currently being used as the mode of delivery for the above-mentioned vaccines as well as for Alnylam Pharmaceuticals' gene silencing therapy, ONPATTRO®, and for Intellia Therapeutics in vivo CRISPR genome editing therapeutic, which has shown for the first time safety and efficacy of in vivo CRISPR genome editing in humans. While nanoparticles and microparticles are safe and effective delivery vehicles, particle synthesis, shipment, and storage can be complicated due to the low temperatures and storage conditions required for the stability of the particles. Similarly, certain small molecule therapeutics in particulate delivery systems may be unstable in solution at ambient temperatures. Thus, storing nanoparticles or microparticles may require storage in a temperature range from −20° C. to −80° C. For example, the storage conditions for nanoparticles are a major impediment to the global roll out of nanoparticle-based SARS-CoV-2 vaccines and is an issue in delivering labile medicines to underserved populations that lack the infrastructure for appropriate refrigeration.

The use of particulate delivery systems for nucleic acids and small molecules, especially with the success of nanoparticle-based vaccines, is certain to grow dramatically along with the associated issues with distribution and storage. Despite the progress made in this area, methods and systems are needed for improved synthesis and administration of particle-based therapeutics that alleviate the supply chain problems with distribution and storage of the particles.

### SUMMARY OF THE INVENTION

The present disclosure relates to methods and systems of synthesizing and administering particle-based (nanoparticle or microparticle) therapeutics (e.g., DNA or RNA encapsulated by lipid nanoparticles) that alleviate the distribution and storage problems associated with conventional particle-based therapeutics. In some embodiments, the present disclosure is related to an apparatus or system that can simultaneously synthesize and administer particle-based therapeutics. For example, the systems described herein can produce nanoparticle-based therapeutics (e.g., nucleic acids adhered to or encapsulated by lipid nanoparticles) in a mixing channel prior to passing the nanoparticle-based therapeutics through a delivery system (e.g., a hypodermic needle) for direct administration to a patient. Described herein are apparatus, methods, and systems capable of synthetizing and administering particle-based therapeutics.

In some embodiments, the present disclosure relates to an apparatus for point-of-care synthesis and administration. The apparatus may include a plurality of chambers comprising a first chamber and a second chamber proximal; an actuator coupled to the first chamber and the second chamber; a mixing channel downstream the first chamber and the second chamber, wherein the first chamber and the second chamber are each in fluid communication with the mixing channel; and a delivery system downstream from the mixing channel, wherein the delivery system is in fluid communication with the mixing channel. In some embodiments, the apparatus further comprises a first channel connecting the first chamber to the mixing channel and a second channel connecting the second chamber to the mixing channel. In some embodiments, the first channel and the second channel converge into a single feed channel to the mixing channel. In some embodiments, the mixing channel comprises a micromixer.

The present disclosure relates to a system for point-of-care synthesis and administration. The system may include a first chamber for receiving a first solution comprising nucleic acids in a first solvent; a second chamber for receiving a second solution comprising lipids in a second solvent; a mixing channel downstream the first chamber and the second chamber for receiving the first solution and the second solution, wherein the mixing channel is in fluid communication with the first chamber and the second chamber for mixing the first solution and the second solution to produce a third solution comprising lipid encapsulated nucleic acids; and a delivery system downstream from the mixing channel, wherein the delivery system is in fluid communication with the mixing channel.

The present disclosure also relates to a method for administering particle-based therapeutics. The method may include providing an apparatus that comprises a first chamber; a second chamber proximal to the first chamber; an actuator coupled to the first chamber and the second chamber; a mixing channel downstream the first chamber and the second chamber, wherein the first chamber and the second chamber are each in fluid communication with the mixing channel; and a delivery system downstream from the mixing channel, wherein the delivery system is in fluid communication with the mixing channel filling the first chamber with a first solution comprising nucleic acids in a first solvent; filling the second chamber with a second solution comprising lipids in a second solvent; applying a force to each of the first chamber and the second chamber to force the first solution and the second solution into the mixing channel; forming lipid encapsulated nucleic acids in the mixing channel; and administering the lipid encapsulated nucleic acids through the delivery system.

### BRIEF DESCRIPTION OF THE DRAWINGS

FIG. 1 illustrates a schematic diagram of an apparatus for synthesizing and administering particle-based therapeutics according to some embodiments.

FIG. 2A illustrates a schematic diagram of another apparatus for synthesizing and administering particle-based therapeutics including a spring and piston system according to some embodiments.

FIG. 2B illustrates a schematic diagram of another apparatus for synthesizing and administering particle-based therapeutics including a dialysis unit according to some embodiments.

FIG. 3 illustrates a schematic diagram of another apparatus for synthesizing and administering particle-based therapeutics including a gas-pressurized piston system according to some embodiments.

FIG. 4 illustrates a schematic diagram of another apparatus for synthesizing and administering particle-based therapeutics including a gas-pressurized system according to some embodiments.

FIG. 5 provides a flow diagram of using an apparatus for synthesizing and administering particle-based therapeutics according to some embodiments.

FIGS. 6A-6H illustrate multiple views of an apparatus for synthesizing and administering particle-based therapeutics according to some embodiments.

FIG. 7 illustrates a schematic diagram of another apparatus for synthesizing and administering particle-based therapeutics including a dialysis unit according to some embodiments.

FIGS. 8A-8C illustrate multiple views of a microfluidic chip for dialysis with counter flow according to some embodiments.

FIG. 9 illustrates a cross-section of a dialysis membrane in a flow channel according to some embodiments.

FIGS. 10A-10C illustrates multiple views of another microfluidic chip for dialysis with parallel flow according to some embodiments.

FIG. 11 illustrates a schematic diagram of an apparatus for administering a therapeutic according to some embodiments.

FIGS. 12A-12D illustrate multiple cross-sectional views of an apparatus for synthesizing and administering particle-based therapeutics according to some embodiments.

### DETAILED DESCRIPTION OF SPECIFIC EMBODIMENTS

The present disclosure describes a number of embodiments related to point-of-care synthesis and administration of particle-based therapeutics. In some embodiments, the present disclosure provides methods and systems that decentralize the synthesis of particle-based therapeutics. This allows for optimized storage and shipment of individual components of particle-based therapeutics with formulation occurring at the point-of-care. For example, a therapeutic such as nucleic acids (e.g., DNA and/or RNA) can be stored and shipped in an aqueous solution (e.g., in vials or cartridges) or as lyophilized powders, while the particle-based components (e.g., lipids) can be stored and shipped in a solution (e.g., an organic solution) or as lyophilized powders for longer-term storage or stockpiling. The particle-based therapeutics (e.g., lipid nanoparticles encapsulating RNA) can be formed at the point of administration by combining the two components, thereby avoiding the stability issues of the particles themselves and minimizing particle aggregation. In some embodiments, the present disclosure provides for the modular synthesis of nucleic acid based therapeutics in which the fixed components of the particles (e.g., lipids and excipients) can be shipped and stored ahead of time, and then novel nucleic acid payloads (e.g., RNA or DNA) can be synthesized independently and combined at the site of administration.

Conventionally, particle components (e.g., nanoparticles or microparticles) and payloads (e.g., nucleic acids, drugs, proteins, etc.) that will be formulated into particle-based therapeutics are synthesized in separate batches. The particle components and payloads are then combined to produce particle-based therapeutics prior to shipping to points of administration (e.g., clinics, hospitals, etc.). For example, the particle-based therapeutic can be nucleic acids or small molecules adhered to or encapsulated by lipid nanoparticles or microparticles. However, shipping the formulated particle-based therapeutics necessitates special storage conditions, which have slowed global accessibility to these therapeutics.

While particle components are safe and effective delivery vehicles, particle synthesis, shipment, and storage can be complicated due to the low temperatures and storage conditions required for the stability of the particles. For example, lipid nanoparticles may require storage in a temperature range from −20° C. to −80° C. to prevent degradation. The storage conditions for lipid nanoparticles are a major impediment to the global roll out of nanoparticle-based vaccines and is an issue in delivering labile medicines to underserved populations that lack the infrastructure for appropriate refrigeration. Additionally, polyethylene glycol is conventionally added to particle-based therapeutics to stabilize particles while in storage, which may result in unwanted side effects and decreased potency of a therapeutic.

Furthermore, personalized vaccines are being developed for cancer therapies which require individual particle-based therapeutics for every patient. However, the time from sequence analysis to mRNA production to particle production needs to be very rapid since cancers can mutate quickly. Additionally, each batch of particles is formulated for a particular individual, which requires manufacture, quality control, shipment, and release of the therapeutic, which is difficult to control and tailor for each individual.

The present disclosure provides a system for synthesizing particle-based therapeutics at the point of administration to avoid stability and storage issues typically associated with drug delivery using nanoparticles. This would allow for decentralized manufacturing of therapeutics (e.g., nucleic acids or small molecules) and regionally specific therapeutics around the world. Since therapeutics such as mRNA are the most labile component of vaccines, manufacturing these components locally would reduce the cost of manufacturing, storage, and shipping. Additionally, the present disclosure provides systems and methods that avoid the use of polyethylene glycol for particle stabilization since the particles can be synthesized at the point of administration. This avoids the negative side effects and potency issues associated with using polyethylene glycol in particle-based therapeutics. Moreover, new or previously unusable lipids and polymers can be considered as particle carriers since long-term storage is no longer needed.

Additionally, the systems and methods described herein provide formulation and administration of personalized mRNA solutions at the point of care. For example, personalized mRNA solutions can be inserted into the apparatus described herein to produce particle-based therapeutics (e.g., mRNA vaccines) at the point of administration. This would eliminate many steps and complications in the manufacturing process for personalized vaccines. This is especially important for rapidly mutating cancers (or mutating viruses) that require timely deployment of vaccines.

In some embodiments, the system includes a first chamber for receiving a lipids in a solvent, a second chamber for receiving a payload (e.g., nucleic acids), a mixing channel in communication with the first chamber and the second chamber, and a delivery system (e.g., a high-gauge hypodermic needle) downstream from the mixing channel. The lipid solution in the first chamber and the payload in the second chamber can be simultaneously introduced into a mixing channel. The particle-based therapeutic can form in the mixing channel and the payload adheres to or is encapsulated by the formed particles to produce a particle-based therapeutic. For example, the mixing channel can form the particle-based therapeutic by hydrodynamic focusing or the mixing channel can include a micromixer to produce the particle-based therapeutic. The formulated particle-based therapeutic can be passed through the delivery system (e.g., a needle) to a subject for administration. The microfluidic system decentralizes the synthesis of injected particle-based therapeutics for optimized storage and shipment of individual components to avoid stability and storage issues, among other advantages described above.

In some embodiments, the system or apparatus for synthesizing particle-based therapeutics does not include a delivery system. For example, the formulated particle-based therapeutic can be fed from the mixing channel directly to a vial. The formulated particle-based therapeutic can be extracted from the vial via a syringe for administration. In some embodiments, a delivery system can be removably attached to the apparatus for synthesizing particle-based therapeutics. In this embodiment, the particle-based therapeutic can be formulated in the mixing channel and then the delivery system (e.g., needle) can be attached to the apparatus for direct administration to a patient. In some embodiments, the system or apparatus may include a separation unit to purify the particle-based therapeutic. In some embodiments, the system or apparatus may include a flow restrictor to between the mixing channel and the delivery system to provide a slow injection rate.

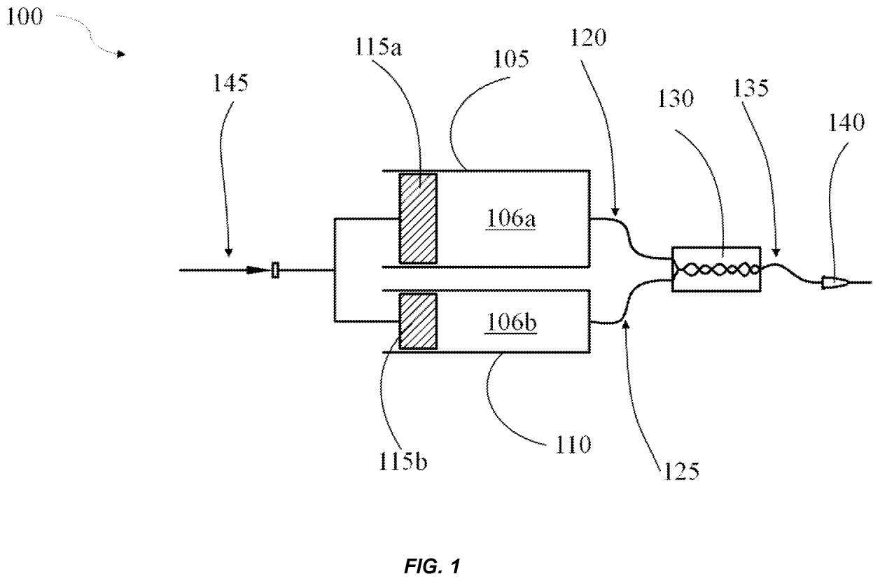

FIG. 1 illustrates a schematic diagram of an apparatus for synthesizing and administering particle-based therapeutics according to some embodiments. The apparatus 100 may include a first chamber 105 and a second chamber 110. The first chamber 105 and the second chamber 110 can be a channel or housing having an interior volume 106a, 106b, respectively. For example, first chamber 105 and the second chamber 110 can be a container having an inlet for receiving a fluid in an interior volume. In some embodiments, each of the first chamber 105 and the second chamber 110 may include a mechanical means for applying a force to the interior volume. As shown in FIG. 1, the first chamber 105 and the second chamber 110 may include a piston 115a, 115b that can be actuated to dispense the contents of the chambers. The contents of the first chamber 105 can be forced into a first channel 120 and the contents of the second chamber 110 can be forced into a second channel 125 by actuating the mechanical means (e.g., piston 115a, 115b) of the first chamber 105 and the second chamber 110. In some embodiments, the piston 115a, 115b is coupled to an actuator 145 that exerts a force onto the piston 115a, 115b to force the solutions in the first chamber 105 and the second chamber 110 through the chambers. In some embodiments, the actuator 145 can be a linear actuator, a hydraulic actuator, a pneumatic actuator, an electrical actuator, or a mechanical actuator. In some embodiments, the actuator 145 can be a spring or pressurized air.

Each of the first chamber 105 and second chamber 110 is configured to receive a volume of fluid. For example, the first chamber 105 may receive a first solution. The first solution may comprise lipids in a first solvent. In some embodiments, the solvent can be an organic solvent. The organic solvent can be an alcohol, for example, methanol, ethanol, propanol (such as isopropanol), butanol (such as n-butanol, isobutanol, sec-butanol, tent-butanol), pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol), dimethyl sulfoxide, n-methyl-2-pyrrolidone, or a combination thereof. In some embodiments, the first solvent is ethanol.

In some embodiments, the lipids may comprise one or more lipids or amphiphilic compounds. For example, the particles can be liposomes, lipid micelles, solid lipid particles, or lipid-stabilized polymeric particles. The lipids can be made from one or a mixture of different lipids. Lipids are formed from one or more lipids, which can be neutral, anionic, cationic, or ionizable. For example, ionizable lipids can be positively charged during production, neutral in storage and in the blood, and revert to positive charge in lysosomes. In some embodiments, ionizable lipids may be composed of an amine moiety and a lipid moiety, and a cationic amine moiety and a polyanion nucleic acid interact electrostatically to form a positively charged liposome or lipid membrane structure. Thus, uptake into cells is promoted and nucleic acids are delivered into cells. It is contemplated by the present disclosure that other types of nanoparticles can be used in the apparatus and system described herein. For example, the apparatus and system described herein can produce or use polymeric nanoparticles, dendrimers, inorganic particles, among others, to produce particle-based therapeutics.

In some embodiments, the apparatus and system described herein can be used to produce, nanoparticle-based therapeutics, microparticle-based therapeutics, or small molecules in suspension. As used herein, “nanoparticles” refer to polymeric particles in the nanometer range. As used herein, “microparticles” refer to polymeric particles in the micrometer size range. For example, the particle-based therapeutic can have a size range from 5 nm to 500 μm, e.g., from 10 nm to 400 μm, from 15 nm to 250 μm, from 20 nm to 200 μm, from 25 nm to 100 μm, or from 30 nm to 200 nm.

The second chamber 110 may receive a second solution comprising a therapeutic as a payload. In some embodiments, the therapeutic may comprise one or more nucleic acids, antigens, peptides, proteins, or small molecules. For example, the second solution may include nucleic acids in a second solvent. The second solvent may comprise water, oils, saline, polyethylene glycols, glycerin, propylene glycol or other synthetic solvents. In some embodiments, the second solution may also include an aqueous buffer solution. The term “nucleic acid” refers to any natural or synthetic linear and sequential arrays of nucleotides and nucleosides, for example, DNA including complementary DNA (cDNA), replicating RNA (repRNA), messenger RNA (mRNA), small interfering RNA (siRNA), transfer RNA (tRNA), microRNA (miRNA), guide strand RNA (sgRNA), polynucleotides, oligo-nucleotides, oligo-nucleosides and derivatives thereof. Such nucleic acids may be collectively referred to as “constructs.” The term “nucleic acid” further includes modified or derivatized nucleotides and nucleosides such as, but not limited to, halogenated nucleotides such as, but not only, 5-bromouracil, and derivatized nucleotides such as biotin-labeled nucleotides. In some embodiments, the apparatus and system described herein can be used to produce particle-based therapeutics including small or large molecules. For example, the apparatus and system described herein could be used to deliver drugs including small or large molecules that are water insoluble or water labile.

The apparatus 100 may include a mixing channel 130 downstream from the first chamber 105 and the second chamber 110. The first chamber 105 and the second chamber 110 are in fluid communication with the mixing channel 130. The mixing channel 130 can be a channel, a microchannel, a micromixer, a mixing device, or combinations thereof. In some embodiments, the mixing channel 130 is a channel that is configured to produce particle-based therapeutics (e.g., lipid nanoparticles encapsulating nucleic acids) using hydrodynamic focusing. In some embodiments, mixing channel 130 may comprise a first region for hydrodynamic focusing and a second region including a micromixer. In other words, mixing channel 130 can use a combination of a mixing channel for hydrodynamic focusing and a micromixer. In some embodiments, the mixing channel 130 is a chamber having a volume for receiving fluids from the first chamber 105 and the second chamber 110.

As shown in FIG. 1, a first solution in the first chamber 105 can be dispensed into a first channel 120 that is fed into the mixing channel 130. Similarly, a second solution in the second chamber 110 can be dispensed into a second channel 125 that is fed into the mixing channel 130. In some embodiments, the first solution and the second solution are not mixed until each solution is provided to the mixing channel 130. Mixing can be carried out by any suitable means known in the art, for example, microfluidic mixing. In some embodiments, the mixing channel 130 can be a micromixer. For example, the micromixer can provide improved mixing by folding of the laminar flow streams, improving diffusion between the two streams and/or turbulent mixing. The micromixer can form the particle-based therapeutic, e.g., nucleic acids bound to or encapsulated in the lipid nanoparticles. In some embodiments, the micromixer can be a passive micromixer (e.g., pressure) or an active micromixer using an external energy source (e.g., electric or magnetic energy).

In some embodiments, the apparatus 100 includes a delivery system 140 downstream from the mixing channel 130. The delivery system 140 may be coupled to the mixing channel 130 via a third channel or tube 135. In some embodiments, the third channel or tube 135 may include a flow restrictor to provide a slow injection rate to the delivery system 140. In some embodiments, the apparatus may not include a delivery system 140 coupled to the mixing channel 130. For example, the formulated particle-based therapeutic can be fed to a vial from the mixing channel 130. The formulated particle-based therapeutic can be extracted from the vial via a syringe for administration. In some embodiments, the delivery system 140 can be removably attached to the apparatus 100. In this embodiment, the particle-based therapeutic can be formulated in the microfluidic system and then the delivery system (e.g., needle) can be attached to the microfluidic system for administration.

In some embodiments, the particle-based therapeutic produced in the mixing channel 130 can be provided to the delivery system 140 for direct delivery to a patient. Examples of delivery systems include, for example, a needle, a catheter, a cannula, a hypodermic needle, a cannula, or a microneedle array. In some embodiments, the delivery system 140 is a hypodermic needle that is downstream from the mixing channel 130. For example, after a lipid-based therapeutic is produced in the mixing channel 130, the therapeutic can be administered to the patient via the hypodermic needle. The apparatus 100 synthesizes and administers the particle-based therapeutic at the point-of-care, thereby avoiding any storage or stability issues associated with formulation and transport.

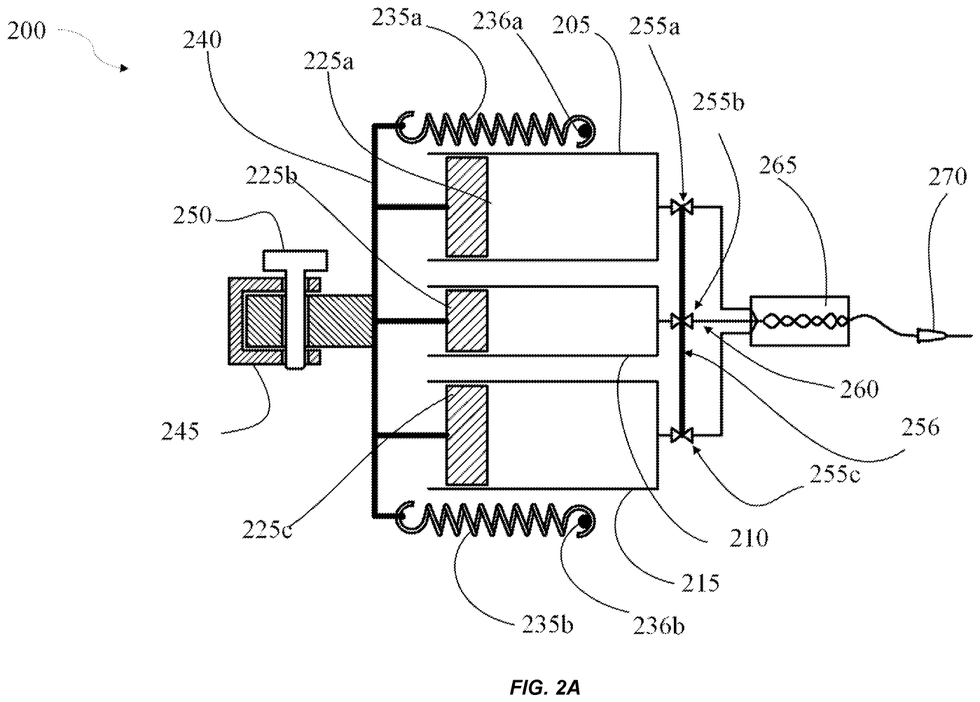

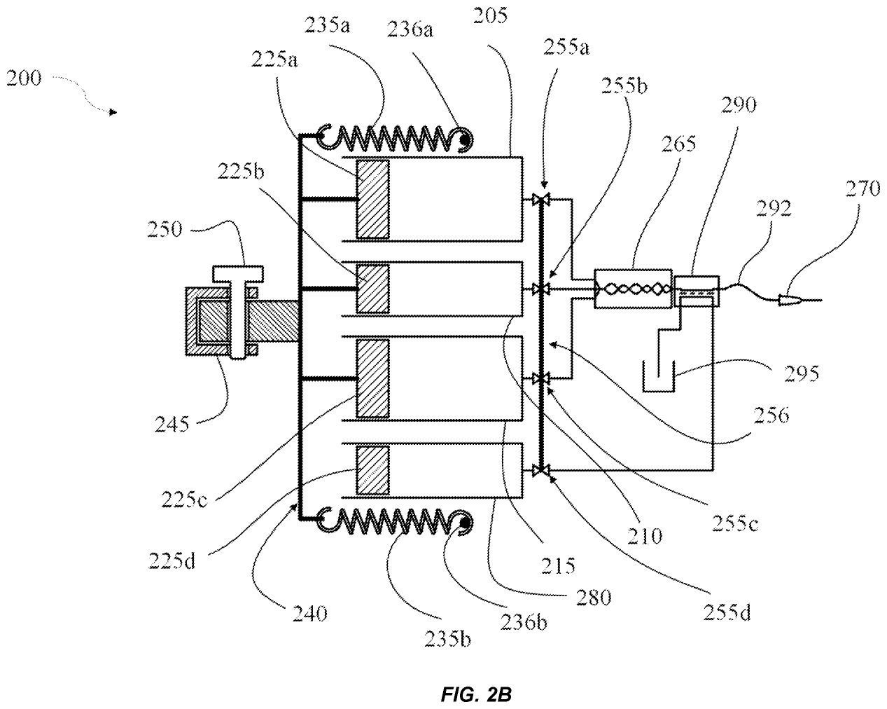

FIG. 2A illustrates a schematic diagram of an apparatus for synthesizing and administering particle-based therapeutics according to some embodiments. The apparatus 200 is a microfluidic system that includes a first chamber 205, a second chamber 210, and a third chamber 215. In some embodiments, the apparatus 200 can include a plurality of chambers. For example, the apparatus 200 can include two or more chambers. In some embodiments, the first chamber 205 is configured to receive a first solution comprising nucleic acids in a first solvent, the second chamber 210 is configured to receive a second solution comprising lipids in a second solvent, and the third chamber 215 is configured to receive a third solution. The third solution may comprise an aqueous solution. For example, the third solution can be a pH modifier. The pH modifier can be used for synthesis of the particle-based therapeutic.

In some embodiments, the third solution in the third chamber 215 is used for hydrodynamic focusing. Hydrodynamic focusing utilizes three streams to pinch off the middle stream, however this can be accomplished by splitting one stream into two separate streams. In some embodiments, the third solution provides an additional component that aids in proper particle formation. This is particularly advantageous for components that cannot be stored with the first solution or the third solution. For example, to produce lipid nanoparticle encapsulated mRNA, the third solution may include HCl to lower the pH to allow the RNA to ionically interact with the ionizable lipid, but HCl (ph from 1.5 to 3.5) would not be directly added to the RNA solution because the maximum stability of RNA is closer to a neutral pH of 6.

The apparatus 200 may include a system for forcing each of the solutions in the first chamber 205, the second chamber 210, and the third chamber 215 downstream to a mixing channel 265 that is coupled directly to a delivery system. As shown in FIG. 2A, the system for forcing each of the solutions through the chambers may include pistons (225a, 225b, and 225c), an anchor 245, and springs 235a, 235b. The apparatus 200 may include pistons 225a, 225b, and 225c disposed in the first chamber 205, the second chamber 210, and the third chamber 215, respectively. The pistons 225a, 225b, and 225c can force the contents of the first chamber 205, the second chamber 210, and the third chamber 215 through each of the chambers to an outlet of the chamber. In some embodiments, pistons 225a, 225b, and 225c can force the contents of the first chamber 205, the second chamber 210, and the third chamber 215 at different flow rates for hydrodynamic focusing in the mixing channel 265.

The pistons 225a, 225b, and 225c can be coupled to a connection structure 240. The springs 235a, 235b are configured to exert a force on the connection structure 240 to move each of pistons 225a, 225b, and 225c. For example, the springs 235a, 235b can exert a force on the connection structure 240 to uniformly move the pistons 225a, 225b, and 225c in each of the chambers. In some embodiments, the springs 235a, 235b can be actuated via an anchor 245. The anchor 245 may include a retaining pin 250. In some embodiments, removing the retaining pin 250 from the anchor 245 will cause the springs 235a, 235b to compress via anchors 236a, 236b and subsequently move the connection structure 240. In some embodiments, retaining pin 250 may be replaced by a latch mechanism. In some embodiments, the apparatus 200 may comprise a dedicated anchor and spring for each of the chambers. In this embodiment, the springs may have different tension forces to force the contents out of the chambers at a flow rate. In some embodiments, the anchor is coupled to springs that have different tension forces to force the contents out of the chamber at different flow rates.

In some embodiments, the pistons 225a, 225b, and 225c may be, for example, configured to move within the cavity of the chamber upon applying pressure to one side of the piston. The chambers may further include a conduit that may be a microfluidic channel or a capillary tube, and that may have a predefined geometry. Upon applying pressure to the pistons 225a, 225b, and 225c within the chamber, a fluid advances within the microfluidic channel at a speed, for example, that may be dictated by the applied pressure, the predefined channel geometry and known fluid properties.

As shown in FIG. 2A, each of the first chamber 205, the second chamber 210, and the third chamber 215 includes an outlet channel (not annotated). Each of the outlet channels from the chambers include valves 255a, 255b, and 255c for regulating the flow of the contents in the first chamber 205, the second chamber 210, and the third chamber 215. In some embodiments, the apparatus 200 includes a valve connection structure 256 which interconnects each of the valves 255a, 255b, and 255c. When closed, the valves 255a, 255b, and 255c prevent the contents in the first chamber 205, the second chamber 210, and the third chamber 215 from flowing through a channel 260 (e.g., a hydrodynamic focusing channel) to a mixing channel 265. When open, the valves 255a, 255b, and 255c allow the contents in the first chamber 205, the second chamber 210, and the third chamber 215 to flow through the channel 260 to the mixing channel 265 (e.g., micromixer) to produce a nanoparticle-based therapeutic. The particle-based therapeutic can be administered using a delivery system 270. The delivery system 270 can be, for example, a needle, a catheter, a hypodermic needle, a cannula, or a microneedle array.

In some embodiments, the mixing channel 265 can be a micromixer. In some embodiments, mixing the contents of the first chamber 205, the second chamber 210, and the third chamber 215 in the mixing channel 265 comprises chaotic advection. In some embodiments, mixing the contents of the first chamber 205, the second chamber 210, and the third chamber 215 comprises mixing with a micromixer. The mixing channel 265 ensures substantially complete mixing of the contents from the first chamber 205, the second chamber 210, and the third chamber 215. In some embodiments, the nucleic acid encapsulation efficiency is from about 80% to about 100% in the mixing channel 265.

In some embodiments, the apparatus 200 may not include a micromixer. In this embodiment, the solutions from each of the first chamber 205, the second chamber 210, and the third chamber 215 converge to single channel 260 to the mixing channel 265 for microfluidic mixing. For example, a first solution from the first chamber 205, a second solution from the second chamber 210, and a third solution from the third chamber 215 can be injected side by side into the channel 260 or the mixing channel 265 at different volumetric flow rates. As the flow streams from the first chamber 205, the second chamber 210, and the third chamber 215 combine in channel 260, mixing and diffusion occurs. The channel 260 can have an appropriate length to ensure complete mixing. In some embodiments, the second solution in the second chamber 210 has a lower volumetric flow rate than the first solution from the first chamber 205 and the third solution from the third chamber 215. This allows hydrodynamic flow focusing of the fluids in the single channel 260 or the mixing channel 265. The use of hydrodynamic flow focusing forms monodisperse lipids of controlled size.

In the basic hydrodynamic flow focusing process, a central solution with a lower flow rate flows within an outer sheath fluid with a higher volumetric flow rate, enabling narrowing of the central flow stream. This narrowing decreases mixing times significantly by reducing the required diffusion length. This process is triggered either by increasing the solute concentration or decreasing the solubility, which results in the formation of nuclei. By controlling the relative flow rates of the solutions, the concentration and solubility can be controlled, which in turn determines the size of the growing nuclei. When the reaction is confined within a focused central flow away from the channel walls, the growing nuclei experience a more uniform solution concentration and spend similar residence time in the mixing channel. Therefore, synthesis in a hydrodynamically-focused stream generates a more homogenous particle size distribution owing to the uniformity of the fluid velocity.

FIG. 2B illustrates another embodiment of the apparatus of FIG. 2A. This embodiment illustrates an apparatus 200 for synthesizing and administering particle-based therapeutics including a separation unit 290. The separation unit 290 is configured to remove waste products (e.g., alcohols) from the particle-based therapeutic. For example, lipids can be supplied to chamber 210 in an ethanol solution. The separation unit 290 can remove ethanol from the particle-based therapeutic. In some embodiments, the separation unit 290 can be a dialysis unit. The dialysis unit can include a membrane that can filter waste products (e.g., alcohols) from the particle-based therapeutic. In some embodiments, the dialysis unit can include a series of membranes that serve as filters. In some embodiments, the membrane can be a reinforced membrane. For example, the membrane can be reinforced with wires, glass fibers, nanotubes, webbings, among others.

The apparatus 200 may include a fourth chamber 280. The fourth chamber 280 may be in communication with the separation unit 290. For example, the fourth chamber 280 may be connected to the separation unit 290 via a tube or channel. The fourth chamber 280 may include a dialysis solution. In some embodiments, the dialysis solution is supplied to the separation unit 290. In some embodiments, the separation unit 290 exchanges the dialysis solution with the particle-based therapeutic to remove alcohol from the particle-based therapeutic. The dialysis solution may comprise a saline solution, phosphate-buffered saline solution, sucrose, among others. The dialysis solution can be an isotonic solution. In some embodiments, the channel or tube 292 connecting the separation unit 290 to the delivery system 270 (e.g., a thin needle) may include a flow restrictor. The flow restrictor may reduce the flow rate of the particle-based therapeutic from the separation unit 290 to the delivery system 270. The flow restrictor can be located in the channel connecting the separation unit 290 to the delivery system 270.

As described in FIG. 2A, the retaining pin 250 can be removed from the anchor 245 to actuate the pistons 225a, 225b, 225c, 225d via the force applied by the springs 235a, 235b on the connecting structure 240. The contents in chambers 205, 210, and 215 are then forced through the mixing channel 265 to produce the particle-based therapeutic. In some embodiments, upon exiting the mixing channel 265, the formulated particle-based therapeutic can be directly passed to a separation unit 290. In some embodiments, the separation unit 290 is a dialysis unit. The particle-based therapeutic may be fed to the separation unit 290 for ion exchange such that one component of the particle-based therapeutic generated in mixing channel 265 is exchanged for another component of the rinse solution. For example, when lipids are provided in an ethanol solution, the separation unit 290 can exchange ethanol from the particle-based therapeutic formed in the mixing channel 265 with a dialysis solution. The particle-based therapeutic is then delivered through delivery system 270. In some embodiments, a waste stream from the separation unit 290 is captured in waste container 295.

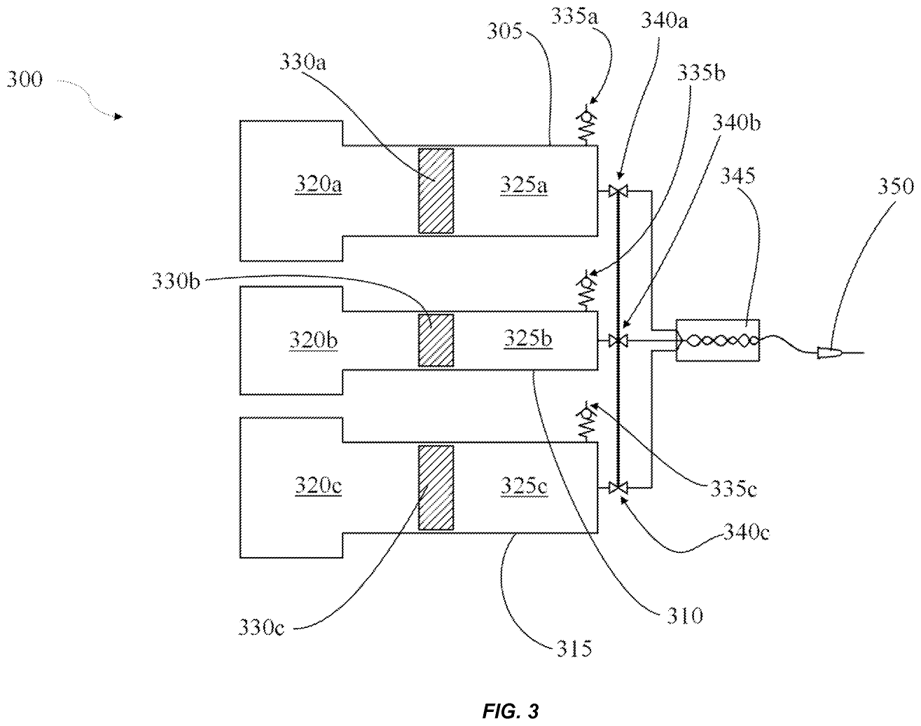

FIG. 3 is another schematic diagram of an apparatus for synthesizing and administering particle-based therapeutics using a gas-pressurized piston system according to some embodiments. In this embodiment, the apparatus 300 utilizes gas pressurization to drive components (e.g., solutions) through each of the first chamber 305, a second chamber 310, and a third chamber 315 to the mixing channel 345. Each of the first chamber 305, the second chamber 310, and the third chamber 315 may each include a first region 320a, 320b, and 320c separated from a second region 325a, 325b, and 325c. Each of the first regions and the second regions of the chambers are separate and comprise an interior volume for receiving fluids. In some embodiments, the first regions 320a, 320b, and 320c may be configured to receive a volume of gas and the second regions 325a, 325b, and 325c may be configured to receive a volume of solution. For example, the first regions 320a, 320b, and 320c may have a volume for receiving compressed gas.

In some embodiments, gas pressure can be used to move or push the components in the chambers through the apparatus 300. As shown in FIG. 3, the first regions 320a, 320b, and 320c can have a volume for receiving pressurized or compressed gas. The first regions 320a, 320b, and 320c may be at atmospheric pressure when the apparatus 300 is not charged or may be at a higher pressure to provide additional driving force. In some embodiments, solutions are loaded into the second regions 325a, 325b, and 325c of the first chamber 305, the second chamber 310, and the third chamber 315 through fill ports (not shown) and check valves 335a, 335b, 335c. Alternatively, check valves 335a, 335b and 335c may each be replaced with a septum. As the first chamber 305, the second chamber 310, and the third chamber 315 are being loaded, moveable pistons 330a, 330b, and 330c are displaced by the solutions. In order to move the pistons 330a, 330b, and 330c to supply the solutions from the first chamber 305, the second chamber 310, and the third chamber 315 to the mixing channel 345, the pressure in first regions 320a, 320b, and 320c need to increase. For example, when the particle-based therapeutic is administered, valves 340a, 340b and 340c are opened and then the gas pressure in second regions 325a, 325b and 325c forces pistons 330a, 330b, and 330c to move and solutions are forced through the chambers to the mixing channel 345. The particle-based therapeutic can be administered using a delivery system 350. The delivery system 350 can be, for example, a needle, a catheter, a hypodermic needle, a cannula, or a microneedle array. In some embodiments, the apparatus 300 includes a flow restrictor. The flow restrictor may reduce the flow rate of the particle-based therapeutic from the mixing channel 345 to the delivery system 350. The flow restrictor can be located in the channel connecting the mixing channel 345 to the delivery system 350.

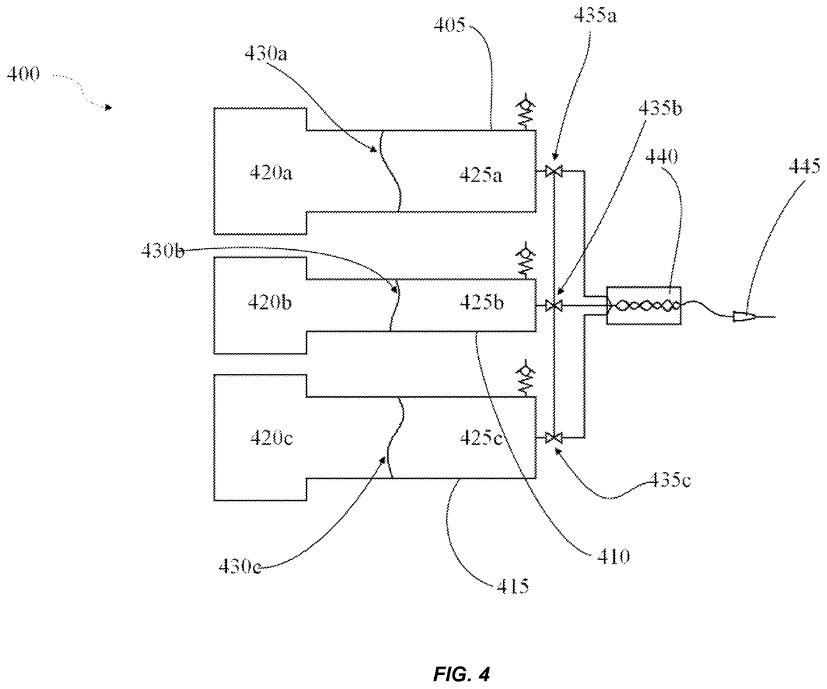

FIG. 4A is another schematic diagram of an apparatus for synthesizing and administering particle-based therapeutics according to some embodiments. In this embodiment, the apparatus 400 utilizes gas pressurization to drive a solution through each of the chambers to the mixing channel or the delivery system. In this embodiment, a mechanical means (e.g., a piston) is not utilized in the first chamber 405, the second chamber 410, or the third chamber 415, however, in some embodiments, a portion of the chambers may utilize a piston. As shown in FIG. 4, the first regions 420a, 420b, and 420c of the chambers may receive a volume of gas that will act directly on the solutions in the second regions 425a, 425b, and 425c of the chambers. The first chamber 405, the second chamber 410, or the third chamber 415 may have an interface 430a, 430b, and 430c between the pressurized gas and the solution in each of the chambers. In some embodiments, pressurized air can be supplied to the first chamber 405, the second chamber 410, and the third chamber 415 to push the solutions through the chambers to the mixing channel 440. The particle-based therapeutic can be produced in the mixing channel 440. For example, when the particle-based therapeutic needs to be synthesized and administered, valves 435a, 435b and 435c are opened and then the gas pressure in the first regions 420a, 420b, and 420c will force the solutions through the mixing channel 440 to a delivery system 445 for administration. The delivery system can be, for example, a needle, a catheter, a hypodermic needle, a cannula, or a micro needle array. In some embodiments, the mixing channel 440 is coupled to a vial for storing the particle-based therapeutic.

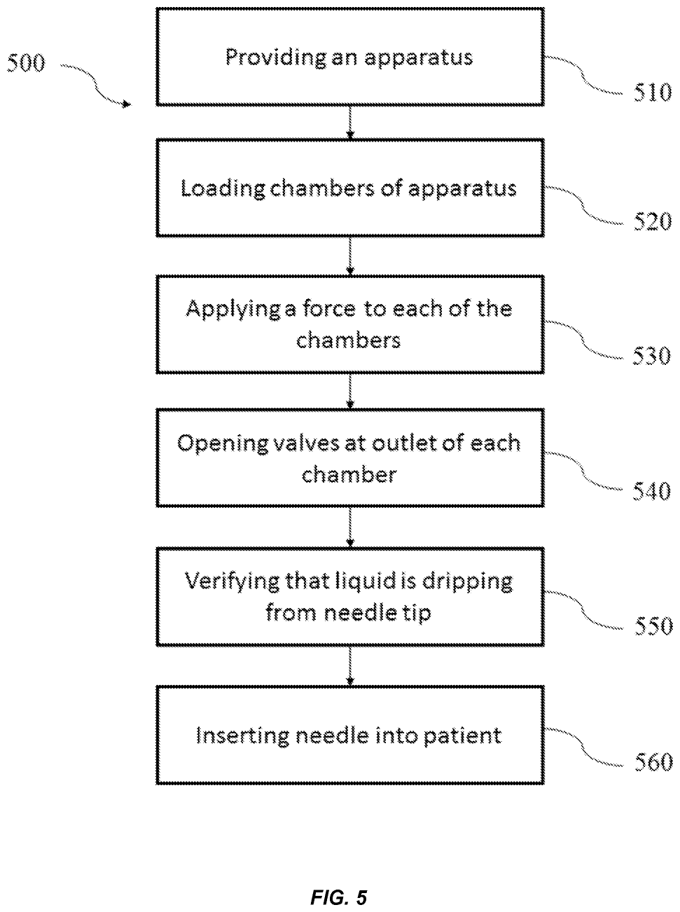

FIG. 5 provides one exemplary method 500 of synthesizing and administering particle-based therapeutics according to embodiments of the present disclosure. The method 500 may include providing 510 an apparatus for synthesizing and administering particle-based therapeutics. For example, the apparatus can be any of the aforementioned devices described in FIGS. 1-4. In some embodiments, the apparatus may include a first chamber for receiving lipids in a first solvent, a second chamber for receiving nucleic acids in a second solvent, a mixing channel in communication with the first chamber and the second chamber, and a delivery system, such as high gauge hypodermic needle, in fluid communication with the mixing channel.

The method 500 may include loading 520 each of the chambers of the apparatus. For example, the method may include filling the first chamber with a first solution. The first solution may include lipids in a second solvent (e.g., ethanol). The method 500 may also include filling the second chamber with a second solution. The second solution may include nucleic acids or small molecules in a second solvent (e.g., an aqueous solution). Each of the first chamber and the second chamber may include a fill port for supplying the first solution and second solution, respectively.

After loading 520 each of the first chamber and the second chamber of the apparatus, the method 500 may include applying 530 a force to each of the first chamber and the second chamber to force the first solution and the second solution through the chambers. For example, each of the chambers may include a piston that is coupled to a connecting structure. The connecting structure is coupled to a spring. An anchor including a retaining pin holds the spring in a static position. In some embodiments, the method includes removing a retaining pin in the anchor to compress the spring and move the pistons in each chamber via the connecting structure.

In some embodiments, the method 500 may include opening 540 valves to force the first solution and second solution to flow from the first chamber and the second chamber to a mixing channel. When the valves are opened, the first solution and the second solution can flow to a mixing channel that is in communication with the first chamber and the second chamber. In some embodiments, the mixing channel may comprise a micromixer. In some embodiments, the mixing channel is capable of hydrodynamic focusing of the first solution and second solution to form nanoparticles. In this embodiment, the apparatus may not include a micromixer.

After applying a force to each of the first chamber and the second chamber of the apparatus and opening the valves, the method 500 may include mixing the first solution and the second solution in the mixing channel to form particle-based therapeutics. In some embodiments, the mixing channel comprises a micromixer. The micromixer may provide turbulent flow to effectively mix the first solution and the second solution. In some embodiments, the method 500 includes verifying 550 liquid flow from the downstream delivery system. The liquid flow from the delivery system can be verified by detecting dripping from the delivery system before insertion into a patient. After the liquid flow is verified, the method 500 includes inserting 560 a needle into a patient for administration of the nanoparticle-based therapeutic. When administration is complete, the apparatus can be discarded, optionally with the needle removed or covered.

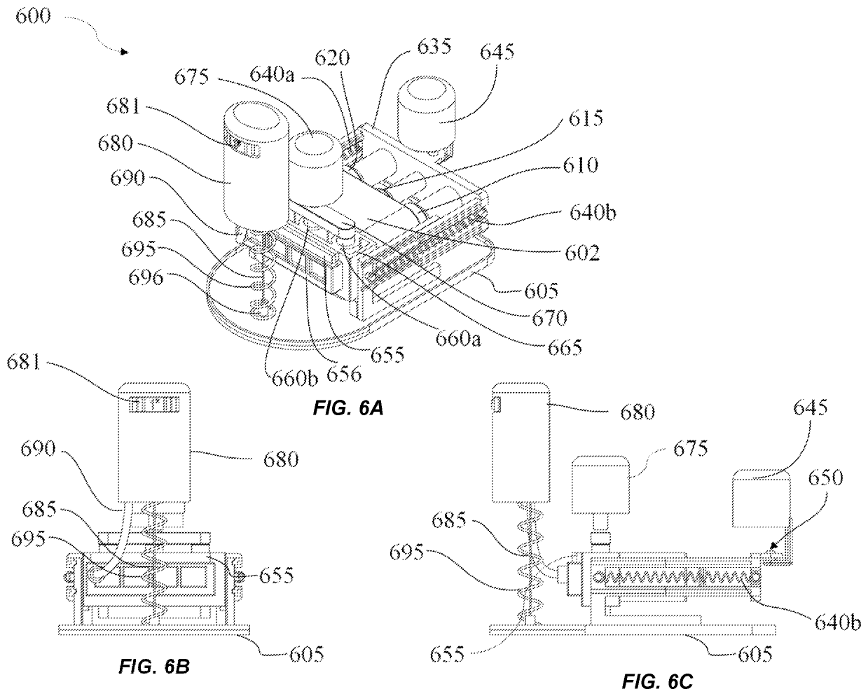

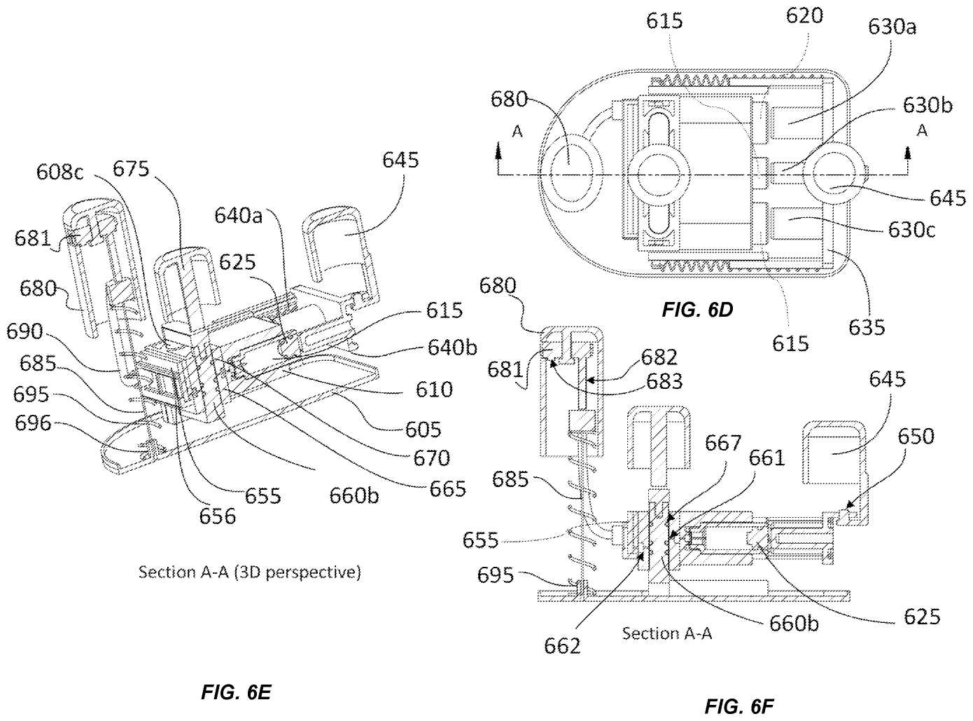

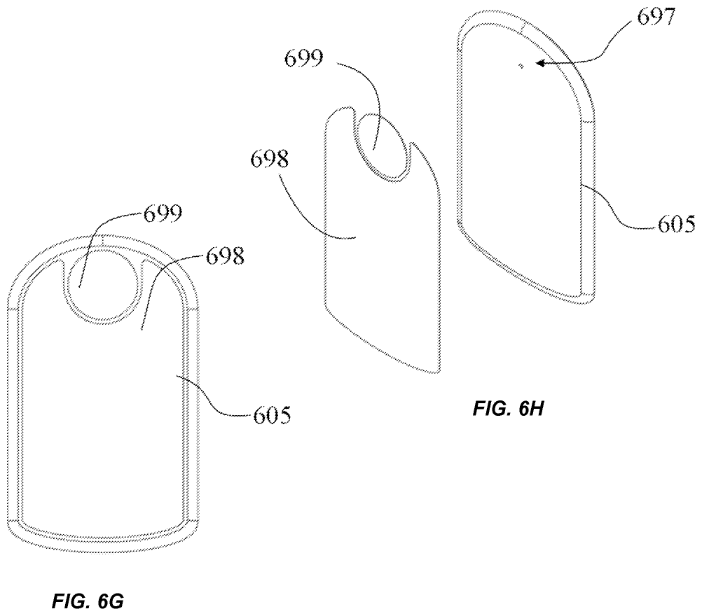

FIGS. 6A-6H illustrate multiple views of another apparatus for synthesizing and administering particle-based therapeutics according to some embodiments. FIG. 6A shows a perspective view of the apparatus 600 for point-of-care particle synthesis and administration, FIG. 6B is a front view of the apparatus 600, and FIG. 6C is a side view of the apparatus 600. FIG. 6D is a top view of the apparatus 600, FIG. 6E is a cross-section of a perspective view of the apparatus 600, and FIG. 6F is cross-section of a side view of the apparatus 600. FIG. 6G is a bottom view of the base plate 605 and FIG. 6H is an exploded perspective view of the base plate 605.

FIG. 6A illustrates an apparatus 600 configured to synthesize and administer a particle-based therapeutic to a patient. The apparatus 600 may include a base plate 605. The base plate 605 of the apparatus 600 may be flat or contoured such that the apparatus can be placed on a surface of a patient for administration of a particle-based therapeutic. When administering a particle-based therapeutic, the base plate 605 of the apparatus 600 can be placed on the arm of the patient, optionally attached with a medical-grade adhesive or wrap.

In some embodiments, the base plate 605 may include a plurality of regions including an adhesive on the surface contacting the patient. For example, as illustrated in FIG. 6G, the base plate 605 may include a first adhesive 698 and a second adhesive 699 in different regions of the base plate 605. FIG. 6H is an exploded perspective view of the base plate 605, the first adhesive 698, and the second adhesive 699. In some embodiments, the adhesive in each region of the base plate 605 may have different compositions. For example, the first adhesive 698 in the first region of the base plate 605 may comprise a medical grade adhesive that is configured to have a stronger adhesive force to the base plate 605 than the skin of the patient. The second adhesive 699 in the second region of the base plate 605 may comprise a medical grade adhesive that is configured to have a stronger adhesive force to the skin of the patient than the base plate 605. The second adhesive 699 in the second region of the base plate 605 may be positioned between the needle aperture 697 and the skin of the patient. During administration of the particle-based therapeutic, a needle deployed through needle aperture 697 can pierce the second adhesive 699. Upon removal of the needle, the second adhesive 699 can remain on the skin of the patient to reduce bleeding from the injection site.

The apparatus 600 may include a main housing 602 disposed on the base plate 605. The housing 602 includes a first chamber 610, a second chamber 615, and a third chamber 620. As shown in FIGS. 6A and 6D, the first chamber 610, second chamber 615, and third chamber 620 may be arranged coaxially in the housing 602. As discussed herein, the first chamber 610 may include a first solution including nucleic acid in a first solvent, the second chamber 615 may include a second solution including lipids in a second solvent, and the third chamber 620 may include a pH modifier solution.

Each of the first chamber 610, second chamber 615, and third chamber 620 may include a plunger 625, as shown in FIG. 6F. FIG. 6F shows a cross-section of a side view of the apparatus 600 showing the plunger 625 within the chamber. FIGS. 6E and 6F only depict the plunger 625 for a single chamber, however, each of the chambers may include a plunger. The plunger 625 may be disposed on one end of the first chamber 610, second chamber 615, and third chamber 620. The plunger 625 is configured to move within the cavity of the chambers when a force is applied to one end of the plunger 625.

As shown in FIG. 6D, the apparatus 600 may include actuators 630a, 630b, and 630c that are configured to move the plunger 625 within the cavity of each of the chambers. As shown in FIG. 6F, the plunger 625 is configured to move within the cavity of the chambers 610, 615, and 620 when pressure is applied to one end of the plunger 625 via the actuators. Each of the actuators 630a, 630b, and 630c can be aligned with the first chamber 610, second chamber 615, and third chamber 620, respectively. The actuators 630a, 630b, and 630c are coupled to a connecting structure 635, as shown in FIG. 6D. The connecting structure 635 can exert a force to move the actuators 630a, 630b, and 630c such that the plungers 625 are pushed through the cavity of each of the chambers.

Referring now to FIG. 6A, a first spring 640a and a second spring 640b are coupled to opposing sides of the connecting structure 635. The apparatus 600 may include a release button 645 (shown in FIG. 6D and FIG. 6E) that includes a retaining pin 650 (shown in FIG. 6F) that is coupled to the first spring 640a and a second spring 640b (shown in FIG. 6A). The release button 645 can be actuated to remove the retaining pin 650 (shown in FIG. 6C) causing the first spring 640a and the second spring 640b to contract. In this way, the first spring 640a and the second spring 640b forces the connecting structure 635 inward to translate each of the actuators 630a, 630b, and 630c towards each of the chambers. Each of the actuators 630a, 630b, and 630c contact and move the plungers 625 within the cavity of the chamber to push a fluid to an outlet of the chambers.

The first chamber 610, the second chamber 615, and the third chamber 620 are in fluid communication with the mixing channel 655. FIG. 6A shows valves 660a, 660b which allows fluid from the first chamber 610 and second chamber 615 to flow to the mixing channel 655. The apparatus may also include a valve for the third chamber 620. The valves 660a, 660b may be disposed in a valve block 665 including a valve connection structure 670. In some embodiments, a valve actuation button 675 acts on the valve connection structure 670 to open or close the valves 660a, 660b. For example, when the valve actuation button 675 is pressed, the actuation force can move the valve connection structure 670 to open or close valves 660a, 660b as shown in FIGS. 6A and 6E. The valves then move downward and allow liquid to flow from the valve inlet 661 to the valve outlet 662 and into the mixing channel 655 (see FIG. 6F). In some embodiments, the valve actuation button 675 and the release button 645 may be connected and actuated simultaneously. In some embodiments, the mixing channel 655 is held against the valve block 665 with a micromixer clamp 656 (shown in FIG. 6A). In some embodiments, O-rings 667 (shown in FIG. 6F) can be used to prevent leakage from the valves 626a, 626b.

As shown in FIGS. 6E and 6F, the mixing channel 655 is in fluid communication with a delivery system 680 comprising a needle 685. For example, a tube 690 (shown in FIGS. 6A and 6E) connects the mixing channel 655 to needle 685 such that fluid can flow from the mixing channel 655 to the needle 685. The delivery system 680 may include a housing with a needle 685 (e.g., a hypodermic needle) extending from a distal end of the housing. In some embodiments, a spring 695 extends from the distal end of the housing of the delivery system 680 and surrounds the needle 685. The depth of needle insertion can be selected with needle depth adjuster 681. For example, FIG. 6F shows the delivery system 680 may include a needle depth adjuster 681 that is coupled to a deep insertion stop 682 and a shallow insertion stop 683. By rotating the needle depth adjuster 681 to the appropriate position, either deep insertion stop 682 or a shallow insertion stop 683 would press on the needle 685 to ensure that the needle 685 is inserted to the correct depth. The spring 695 holds the needle 685 in the retracted position until the apparatus 600 is ready to administer the particle-based therapeutics. A needle guide 696 (shown in FIGS. 6A and 6E) may be provided adjacent to the base plate 605. The needle guide 696 helps to stabilize the needle 685 when it is inserted into the patient. In some embodiments, an additional latch can be used to hold the needle 685 in place and prevent the spring 695 from retracting the needle 685 prematurely.

In some embodiments, the mixing channel 655 is in a vertical orientation relative to the skin of the subject receiving the particle-based therapeutic. In some embodiments, the mixing channel 655 may be oriented such that the mixing channel 655 is in close proximity to the skin of the patient receiving the particle-based therapeutic, thereby ensuring that the temperature of the mixing channel 655 remains approximately similar to the temperature of the patient. This may result in an improved consistency of the properties of the particle-based therapeutic being administered. This configuration may also improve patient comfort during administering of the particle-based therapeutic.

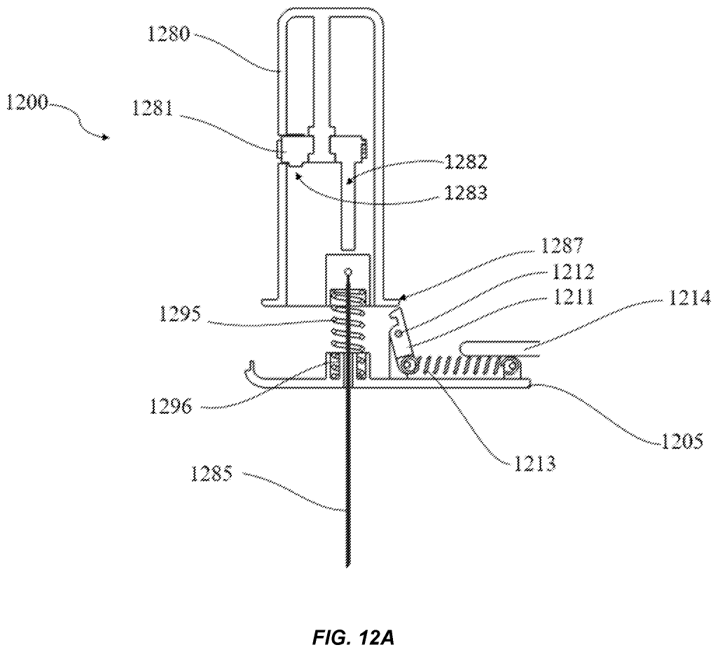

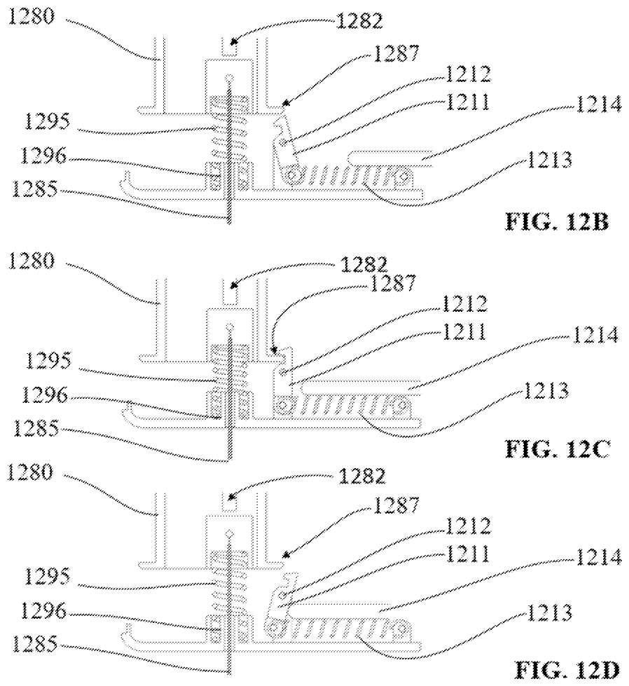

FIGS. 12A to 12D illustrate multiple cross-sectional views of another apparatus for synthesizing and administering particle-based therapeutics according to some embodiments. Specifically, FIGS. 12A to 12D provide an embodiment of the apparatus including a latch mechanism. The latch mechanism may be configured to release a puncturing device (e.g., a needle) after a particle-based therapeutic is administered to a patient. The latch mechanism can be incorporated into any of the embodiments of the apparatus described herein. It is contemplated that any type of latch mechanism can be used in the apparatus, for example, latches with a pivot point, cam latches, compression latches, slam latches, draw latches, snap latches, or sliding latches.

Referring to FIG. 12A, the delivery system 1280 performs a similar function to the delivery system 680 described in FIGS. 6A-6H. The apparatus 1200 may include a delivery system 1280 for deploying a needle 1285 to a desired depth. In some embodiments, the delivery system 1280 may include a needle depth adjuster 1281 that is coupled to a deep insertion stop 1282 and a shallow insertion stop 1283. The needle depth adjuster 1281 is configured to be adjusted (e.g., rotated) to select one of the deep insertion stop 1282 or the shallow insertion stop 1283. For example, by rotating the needle depth adjuster 1281 to the appropriate position, either the deep insertion stop 1282 or the shallow insertion stop 1283 would act on the needle 1285 to ensure that the needle 1285 is inserted to the correct depth. The spring 1295 holds the needle 1285 in the retracted position until the apparatus 1200 is ready to administer the particle-based therapeutic. A needle guide 1296 may be provided adjacent to the base plate 1205. The needle guide 1296 helps to stabilize the needle 1285 when it is inserted into the patient. The latch 1211 is configured to hold the delivery system 1280 and needle 1285 in position while the particle-based therapeutic is being administered to a patient. In this embodiment, the latch 1211 can rotate around pivot point 1212. In some embodiments, the latch 1211 can be held in position by spring 1213. The spring 1213 may engage the catch surface 1287 of delivery system 1280. In some embodiments, pusher rod 1214 is attached to a connecting structure performing a similar function to connecting structure 635 described in FIG. 6A.

FIGS. 12B-12D illustrate the operation of the latch 1211 of the apparatus 1200. FIG. 12B provides a first configuration of the delivery system 1280 and the latch 1211. In the first configuration, the needle 1285 from the delivery system 1280 is almost fully deployed (e.g., the tip of the needle 1285 extends well past the base plate 1205), but the latch 1211 is not engaged with the delivery system 1280. FIG. 12C shows a second configuration of the delivery system 1280 and the latch 1211. In the second configuration, the needle 1285 from the delivery system 1280 is fully deployed and the latch 1211 is engaged with the catch surface 1287 of delivery system 1280. In the second configuration, the latch 1211 is holding the needle 1285 in position while the particle-based therapeutic is being administered to a patient. FIG. 12D shows a third configuration. In the third configuration, the pusher rod 1214 engages with the latch 1211. The force exerted by the pusher rod 1214 on latch 1211 overcomes the restoring force applied by the spring 1213 on the latch 1211, therefore causing the latch 1211 to rotate and disengage from catch surface 1287. This allows the spring 1295 to exert a force on delivery system 1280, thereby retracting needle 1285.

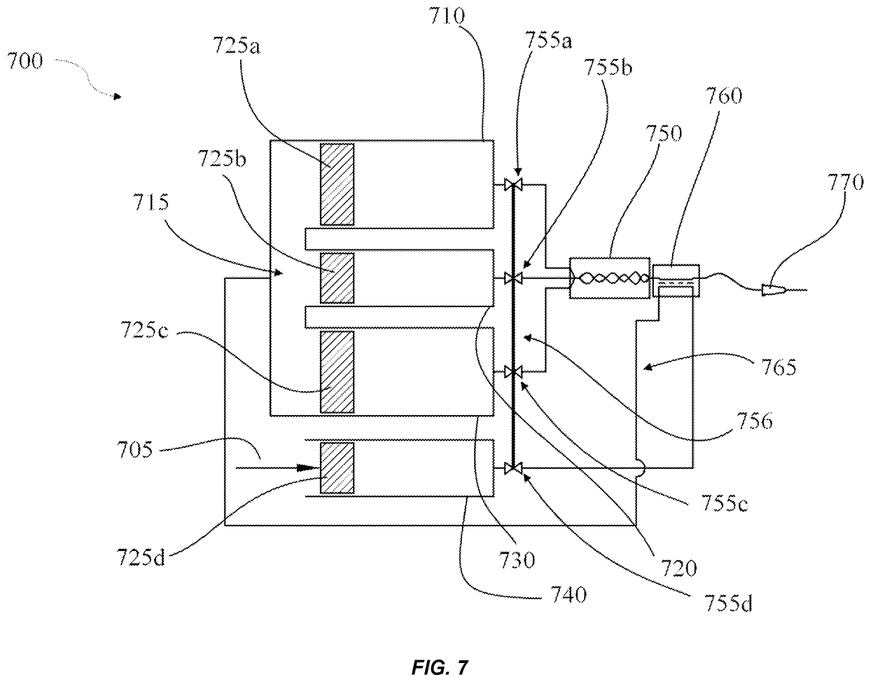

FIG. 7 provides another embodiment of an apparatus for synthesizing and administering a particle-based therapeutic. The apparatus 700 may include a first chamber 710, a second chamber 720, a third chamber 730, and a fourth chamber 740. In some embodiments, the first chamber 710 may receive a first solution comprising nucleic acids and water, the second chamber 720 may receive a second solution comprising lipids in alcohol (e.g., ethanol), the third chamber 730 may receive a third solution comprising a pH modifier, and the fourth chamber 740 may receive a fourth solution comprising a dialysis solution. In some embodiments, the volume of the fourth chamber 740 may be equivalent to the total volume of the first chamber 710, the second chamber 720, and the third chamber 730.

In some embodiments, each of the chambers may include pistons 725a, 725b, 725c, 725d. The pistons 725a, 725b, 725c, 725d are configured to force the contents of the first chamber 710, the second chamber 720, the third chamber 730, and the fourth chamber 740 into an outlet channel. Each of the first chamber 710, the second chamber 720, and the third chamber 730, and the fourth chamber 740 may include an outlet channel comprising valve 755a, 755b, 755c, 755d. Each of the valves 755a, 755b, 755c, 755d from the first chamber 710, the second chamber 720, the third chamber 730, and the fourth chamber 740 may be interconnected via a valve connection structure 756.

The apparatus 700 includes a mixing channel 750 in fluid communication with the first chamber 710, the second chamber 720, and the third chamber 730. A separation unit 760 is in fluid communication with the mixing channel 750. The fourth chamber 740 may supply a dialysis solution to the separation unit 760. For example, the dialysis solution can be a dialysis buffer (e.g., phosphate-buffered saline or normal saline). In some embodiments, the solution comprising the particle-based therapeutic produced in the mixing channel 750 is passed through the separation unit 760 and is then administered to a patient using a delivery system 770 (e.g., a needle). The dialysis solution from the fourth chamber 740 is supplied to the separation unit 760 for exchange with the solution comprising the particle-based therapeutic. For example, the separation unit 760 may include a membrane to exchange the dialysis solution with the solution comprising the particle-based therapeutic. The membrane may prevent the formulated particles from passing, but other components may pass through the membrane for exchange with the dialysis solution. For example, the solutions in each of the first chamber 710, the second chamber 720, and the third chamber 730 may be exchanged with the dialysis solution to produce particle-based therapeutic in the dialysis solution for administration. In other words, components such as ethanol, water, and pH modifiers can be removed from the particle-based therapeutic before administration. The separation unit 760 may also pH neutralize the particle-based therapeutic prior to administration by performing this exchange. In some embodiments, the separation unit 760 can be a counter flow or parallel flow dialysis unit. In some embodiments, the dialysis solution may comprise a saline solution, phosphate-buffered saline solution, sucrose, among others. In some embodiments, the dialysis solution may be isotonic or non-isotonic. In some embodiments, the dialysis solution may act to reduce the volume of the delivered solution including the particle-based therapeutic. For example, a hypertonic dialysis solution may be used to reduce the volume of the delivered solution including the particle-based therapeutic.

The apparatus 700 may include a pressurization chamber 715. The pressurization chamber 715 may include the first chamber 710, the second chamber 720, and the third chamber 730. The interior volume of the pressurization chamber 715 is separated from the interior volume of the first chamber 710, the second chamber 720, and the third chamber 730 by the pistons 725a, 725b, 725c, In some embodiments, a portion of the interior volume of the pressurization chamber 715 is filled with a liquid or is pressurized with gas. In some embodiments, the volume of the fourth chamber 740 may be equivalent to the total volume of the pressurization chamber 715, the first chamber 710, the second chamber 720, and the third chamber 730.

In this embodiment, an external force 705 is applied to the piston 725d in the fourth chamber 740. The external force 705 can be generated by the aforementioned systems (e.g., springs, gas pressurization, etc.) described herein. The dialysis solution in the fourth chamber 740 is forced through an outlet channel to the separation unit 760. In some embodiments, valve connection structure 756 is configured to open each of valves 755a, 755b, 755c, and 755d to begin the process of synthesizing and administering the particle-based therapeutic. The external force 705 applied to piston 725d in the fourth chamber 740 forces dialysis solution through separation unit 760. In some embodiments, the dialysis solution is supplied to an end of the separation unit 760 adjacent to the delivery system 770 for counter flow with the particle-based therapeutic. In this embodiment, the separation unit 760 performs counter flow dialysis.

In operation, each of valves 755a, 755b, 755c, and 755d can be opened by actuating the valve connection structure 756. The dialysis solution is forced through the fourth chamber 740 by applying an external force 705 to the piston 725d to drive the dialysis solution to the separation unit 760. In some embodiments, an external force is only applied to the fourth chamber 740. The dialysis waste stream from separation unit 760 is then fed through return line 765 into pressurization chamber 715. The pressure generated in pressurization chamber 715 by the dialysis waste stream is then used to actuate pistons 725a, 725b, 725c to move the contents of the first chamber 710, the second chamber 720, and the third chamber 730 into an outlet channel to the mixing channel 750 to produce the particle-based therapeutic. Upon exiting the mixing channel 750, the solution comprising the particle-based therapeutic is passed through separation unit 760. In separation unit 760, exchange takes place and one or more components of the solution comprising the particle-based therapeutic generated in mixing channel 750 is exchanged for the dialysis solution. The final particle-based therapeutic (e.g., particle-based therapeutic in buffer solution) is then delivered to a patient through delivery system 770. Although a separation unit is only shown and described in FIG. 2B and FIG. 7, any of the embodiments of the present disclosure may include a separation unit.

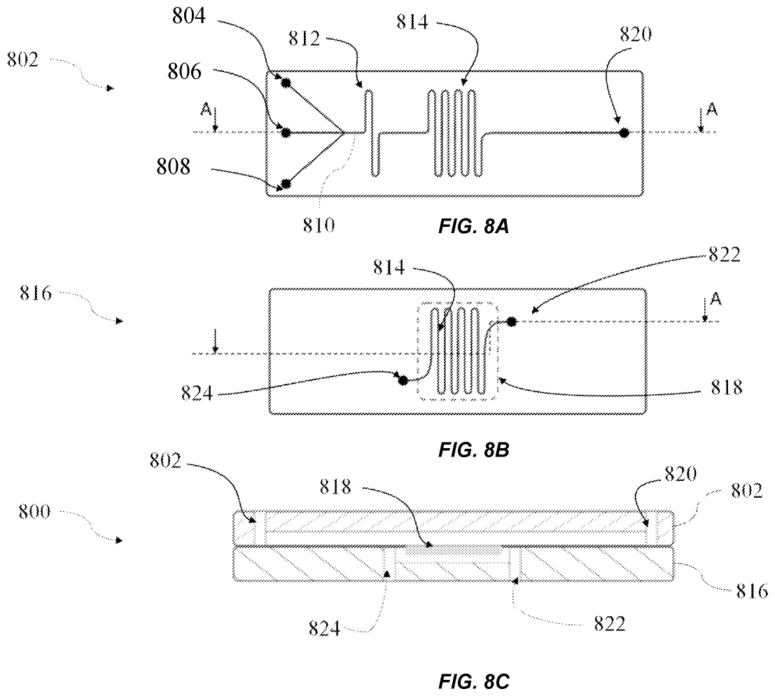

FIGS. 8A-8C shows multiple views of a microfluidic chip according to some embodiments. FIG. 8A is a top cross-sectional view of a top chip, FIG. 8B is a top cross-sectional view of a bottom chip, and FIG. 8C is a cross-sectional view of the top and bottom chips bonded together. In some embodiments, the microfluidic chip 800 can combine the mixing channel and the separation unit described herein. For example, solutions from each of the chambers can be fed to the microfluidic chip 800 to form the particle-based therapeutic in a mixing region followed by separation in a dialysis region.

FIG. 8A shows a top cross-sectional view of a top chip of the microfluidic chip and The microfluidic chip 800 includes a top chip 802 shown in FIG. 8A and a bottom chip 816 shown in FIG. 8B. As shown in the FIG. 8C, the top chip 802 and the bottom chip 816 can be bonded at a bond interface to form the microfluidic chip 800.

In embodiments including three chambers, streams 804, 806, and 808 from each of the chambers can be fed to the microfluidic chip 800. The streams 804, 806, and 808 can converge into a single feed channel 810 to a mixing region 812. In some embodiments, hydrodynamic focusing can occur in the single feed channel 810 to form the particle-based therapeutic. The mixing region 812 may include a micromixer to form the particle-based therapeutic. The dialysis region 814 is in fluid communication with the mixing region 812. The formulated particle-based therapeutic from the mixing region 812 can be passed to the dialysis region 814 to purify the particle-based therapeutic.

FIG. 8B shows a top cross-sectional view of a bottom chip 816 of the microfluidic chip. In some embodiments, the particle-based therapeutic produced in the mixing region 812 is passed through the dialysis region 814 and is then administered to a patient using a delivery system (e.g., a needle). As shown in FIG. 8C, the dialysis region 814 may be partially located in the bottom chip 816. The dialysis region 814 may be a counter current flow dialysis chamber. In some embodiments, the dialysis region 814 includes a membrane 818. The membrane 818 can be a semi-permeable film or a dialysis membrane. In some embodiments, the membrane prevents larger molecules (e.g., the particle-based therapeutic) from passing through the membrane but small molecules can pass through freely. In some embodiments, a dialysis solution is provided to the dialysis region 814 for counterflow with the solution comprising the particle-based therapeutic. As shown in FIG. 8B, the dialysis solution enters the dialysis region 814 through inlet 822 and passes through the dialysis region 814 to outlet 824. At outlet 824, the dialysis waste stream leaves the dialysis region 814. The dialysis waste stream may include components from streams 804, 806, and 808.

The dialysis region 814 exchanges the dialysis solution with components in the solution comprising the particle-based therapeutic. The membrane 818 may prevent the formulated particles from passing, but other components may pass through the membrane for exchange with the dialysis solution. For example, components such as ethanol, water, and pH modifiers may be exchanged with the dialysis solution to purify the particle-based therapeutic before administration. The dialysis region 814 may also pH neutralize the particle-based therapeutic prior to administration by performing this exchange. The purified particle-based therapeutic may be provided to a vial or delivery system via an outlet 820.

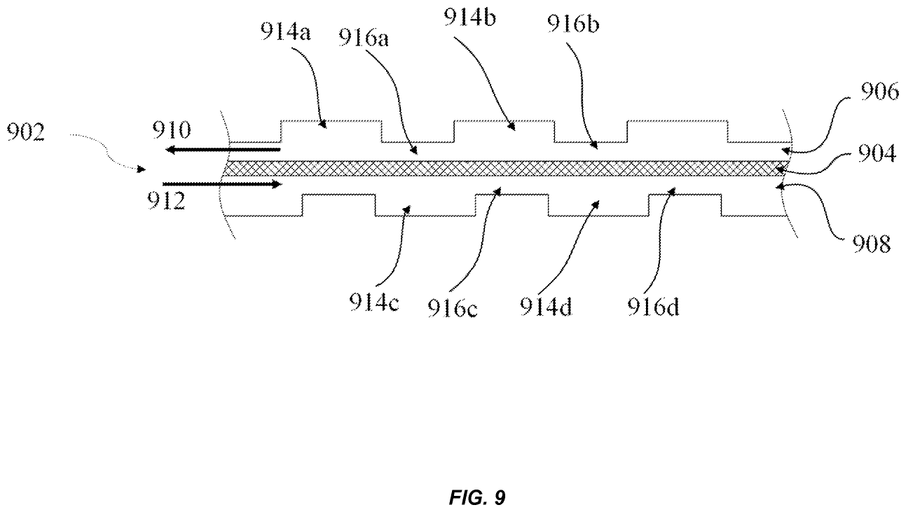

FIG. 9 shows a cross-sectional view of a dialysis membrane in a counter flow dialysis region according to some embodiments. In some embodiments, the microfluidic chip of FIGS. 8A-8C may include the membrane shown in FIG. 9. In some embodiments, the dialysis region may include a flow channel 902 including a membrane 904 separating a first region 906 from a second region 908. In some embodiments, a first solution comprising a particle-based therapeutic may flow through the first region 906 in a first direction 910 and a second solution comprising a dialysis solution may flow through the second region 908 in a second direction 912 counter to the first direction 910.

The flow channel 902 may comprise large channel sections 914a, 914b alternating with small channel sections 916a, 916b in the first region 906. Similarly, the flow channel 902 may comprise large channel sections 914c, 914d alternating with small channel sections 916c, 916d in the second region 908. The large channel sections 914a, 914b in the first region 906 may be opposite the small channel sections 916c, 916d in the second region 908. Similarly, the small channel sections 916a, 916b in the first region 906 may be opposite the large channel sections 914c, 916d in the second region 908. The large channel sections are opposite the small channel sections to restrict and expand flow on different sides of the membrane 904. The successive transition from small channels to large channels (e.g., constriction regions) facilitate mixing and circulation of the dialysis solution and solution including the particle-based therapeutics. One of the challenges with microfluidic systems is that the Reynolds numbers are often relatively small, and therefore the flow is often laminar. Laminar flow is not conducive to good mixing. Therefore, large and small channel sections in each of the first region 906 and the second region 908 can improve mixing by creating recirculation regions. Furthermore, the constriction regions will experience a larger pressure drop per unit length than the expansion regions. The larger channel sections opposing the small channel sections provide constriction regions that may help create a pressure gradient across the membrane and facilitate trans-flow filtration. The trans-flow filtration can also be enhanced by the larger channel sections on the other side of the membrane as shown in this embodiment, thereby increasing the pressure gradient and facilitating the movement of liquid from one side of the membrane to the other. In embodiments including counter flow dialysis, the fluid transfer in either direction facilitates efficient buffer exchange.

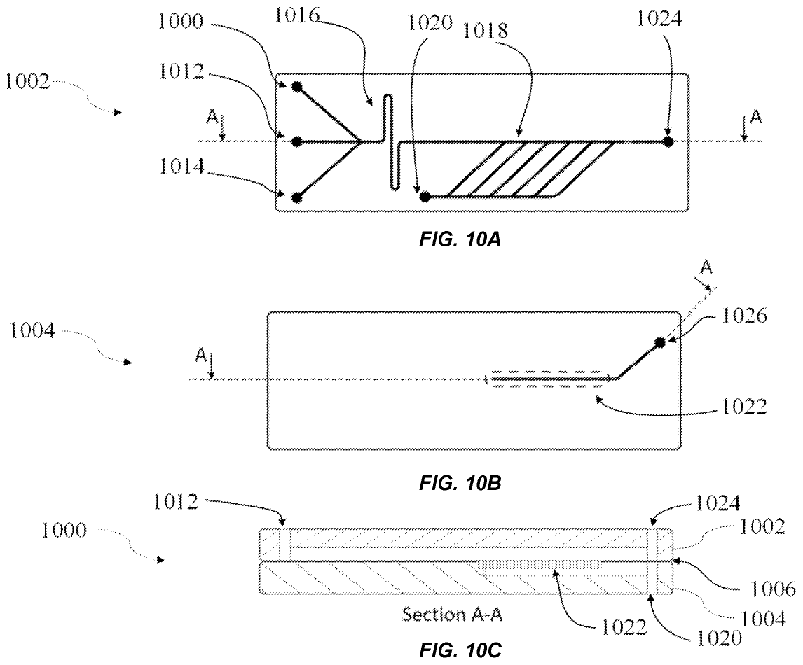

FIGS. 10A-10C shows multiple views of a microfluidic chip including a dialysis region having parallel flow according to some embodiments. FIG. 10A is a top cross-sectional view of a top chip, FIG. 10B is a top cross-sectional view of a bottom chip, and FIG. 10C is a cross-sectional view of the top and bottom chips bonded together. The microfluidic chip 1000 includes a top chip 1002 shown in FIG. 10A and a bottom chip 1004 shown in FIG. 10B. As shown in the FIG. 10C, the top chip 1002 and the bottom chip 1004 can be bonded at a bond interface 1006 to form the microfluidic chip 1000.

As shown in shown in FIG. 10A, streams 1008, 1010, and 1012 can be fed to the microfluidic chip 1000. The streams 1008, 1010, and 1012 can converge into a single feed channel to a mixing region 1016 to form the solution comprising the particle-based therapeutic. The solution comprising the particle-based therapeutic can be passed to a dialysis region 1018 to purify the particle-based therapeutic. The dialysis region 1018 can be in fluid communication with the mixing region 1016. In some embodiments, a dialysis solution can be fed to the dialysis region 1018 through inlet 1020. The dialysis solution can be provided through the dialysis region 1018 in parallel flow with the solution comprising the particle-based therapeutic. In some embodiments, a plurality of flow lines can supply the dialysis solution to the dialysis region 1018 at multiple points.

As shown in FIG. 10B, a portion of the dialysis region 1018 may include a membrane 1022. As described above, the membrane 1022 can be semi-permeable to filter components from the solution containing the particle-based therapeutic. For example, FIG. 10C shows the membrane 1022 disposed between the interface 1006 of the top chip 1002 and the bottom chip 1004. As the particle-based therapeutic flows through the dialysis region 1018, the dialysis solution is passed in parallel in the bottom chip 1004 with a membrane 1022 separating the two streams. The dialysis solution enters the dialysis region 1018 through inlet 1020 and passes through the dialysis region 1018 to an outlet 1026. At the outlet 1026, the dialysis waste stream leaves the dialysis region 1018. The dialysis waste stream may include components from streams 1008, 1010, and 1012. The dialysis region 1018 provides a purified particle-based therapeutic including the dialysis solution at outlet 1024.

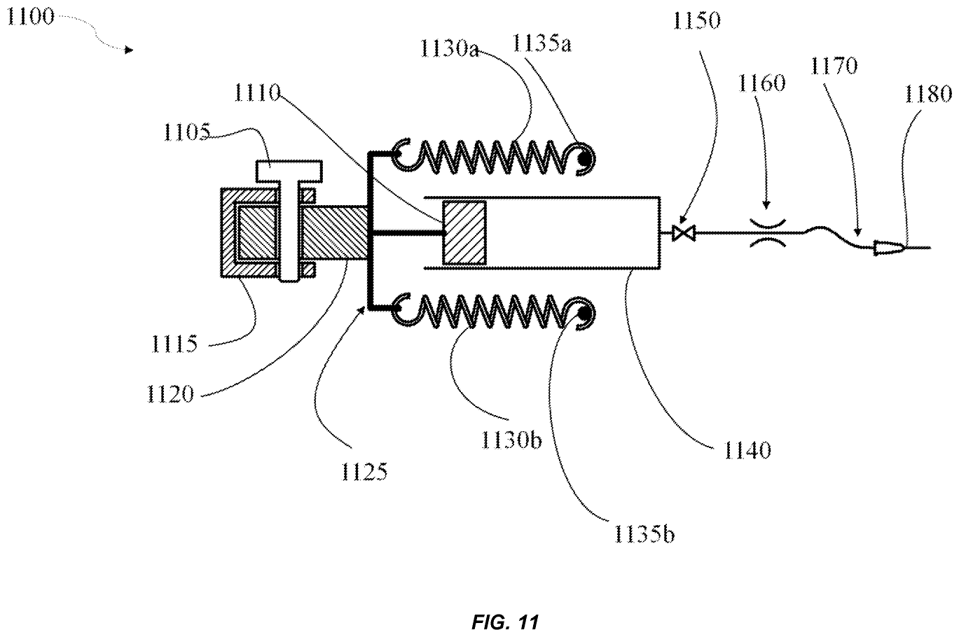

FIG. 11 illustrates a schematic diagram of an apparatus for administering a therapeutic according to some embodiments. In some embodiments, any of the aforementioned embodiments described herein may be used to formulate a therapeutic. For example, the apparatus of FIG. 1 including a plurality of chambers and a mixing channel can be used to formulate a particle-based therapeutic. The apparatus 1100 of FIG. 11 can be used to administer the particle-based therapeutic to a patient. The apparatus 1100 may administer any therapeutic in solution. For example, the apparatus 1100 can administer a therapeutic in solution or a particle-based therapeutic in solution.

In the embodiment shown in FIG. 11, the apparatus 1100 includes a single chamber 1140 for receiving a therapeutic in solution. The apparatus 1100 includes an optional valve 1150 at an outlet channel 1170 from the chamber 1140. The outlet channel 1170 may be in fluid communication with the delivery system 1180. The valve 1150 prevents the contents of the chamber 1140 from being spilled or contaminated. In operation, the valve 1150 may be opened to allow the contents of the chamber 1140 to flow to an outlet channel 1170 to the delivery system 1180.

In some embodiments, the chamber 1140 includes a piston 1110 to force the contents of the chamber 1140 into the outlet channel 1170. For example, an external force is applied to the piston 1110 in the chamber 1140 to force the contents of the chamber 1140 into the outlet channel 1170. For example, similar to the previous embodiments described herein, the piston 1110 is connected to a connecting structure 1125. The apparatus 1100 may include springs 1130a and 1130b that act on connecting structure 1125 that in turn acts on the piston 1110 to apply a force or pressure to the contents of the chamber 1140. The connecting structure 1125 may connected to an anchor 1115 including a retaining pin 1105 via a connection structure 1120.

In some embodiments, the apparatus 1100 includes a flow restrictor 1160. The flow restrictor 1160 may reduce the flow rate of the therapeutic from the chamber 1140 to the delivery system 1180. The flow restrictor 1160 can be located in the channel 1170 connecting the chamber 1140 to the delivery system 1180. The apparatus 1100 can provide a slower injection rate for administering a therapeutic by utilizing the flow restrictor 1160. The slower injection rate may result in less pain to the patient and may result in improved uptake and perfusion of the therapeutic. In some embodiments, the flow restrictor 1160 in conjunction with the delivery system 1180 may reduce the flow rate of the therapeutic. For example, the delivery system 1180 may include a thin needle that reduces the flow rate of the therapeutic. The thin needle may result in less pain during insertion of the needle. In some embodiments, the flow restrictor 1160, the outlet channel 1170, and delivery system 1180 may be combined into the delivery system 1180. By appropriate sizing of the flow restrictor 1160, the outlet channel 1170, and the delivery system 1180 (e.g., needle), a suitable amount of flow resistance is created to result in a slower injection of the therapeutic into the patient. In some embodiments, the injection may be intramuscular, subcutaneous, or intravenous.

In some embodiments, the invention provides a method for producing particle-based therapeutics containing a nucleic acid including introducing a first stream comprising a nucleic acid in a first solvent (e.g., ethanol) into a microfluidic device. The device has a first region adapted for flowing one or more streams introduced into the device and a second region for mixing the contents of the one or more streams with a microfluidic mixer. In some embodiments, the first stream comprises lipids in a first solvent. For example, lipids can be dispersed in an aqueous media to form liposomes (e.g., unilamellar and/or multilamellar liposomes). Liposomes can effectively encapsulate and deliver a wide range of chemicals including nucleic acids, proteins, and small drug particles, to cells. For example, cationic liposomes formed from a composition of cationic lipids and phospholipids form aggregates with anomic macromolecules such as DNA and RNA.

The method includes introducing a second stream comprising nucleic acids in a second solvent (e.g., an aqueous solution) into the device. The first region of the device is adapted for flowing the second stream into the mixing channel and directing the second stream into a second region for mixing the contents of the first stream and the second stream. In some embodiments, the first and second solvents are not the same. In some embodiments, the first stream and the second stream are maintained in physical separation in the first region. For example, the one or more first streams and the one or more second streams do not mix until arriving at the second region of the channel.

The method includes flowing the one or more first streams and the one or more second streams from the first region of the device into the second region of the device. The contents of the one or more first streams and the one or more second streams are mixed in the second region of the device to provide a third stream comprising lipid nanoparticles with encapsulated nucleic acid. The method includes flowing lipid nanoparticles with encapsulated nucleic acid to a downstream delivery device. In some embodiments, the delivery device is a needle, a catheter, a hypodermic needle, a cannula, or a microneedle array. The delivery device can be used to directly administer the synthesized lipid particles with encapsulated nucleic acid to a patient.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It will be apparent to one of ordinary skill in the art that many modifications and variations are possible in view of the above teachings.

1. An apparatus for point-of-care administration, the apparatus comprising:
a plurality of chambers comprising a first chamber and a second chamber;
an actuator coupled to the first chamber and the second chamber;
a mixing channel downstream the first chamber and the second chamber, wherein the first chamber and the second chamber are each in fluid communication with the mixing channel; and
a delivery system downstream from the mixing channel, wherein the delivery system is in fluid communication with the mixing channel.

2. The apparatus of claim 1, wherein the apparatus further comprises:
a first channel connecting the first chamber to the mixing channel; and
a second channel connecting the second chamber to the mixing channel;
wherein the first channel and the second channel converge into a single feed channel to the mixing channel.

3. (canceled)

4. The apparatus of claim 1, wherein the mixing channel comprises a micromixer.

5. The apparatus of claim 1, wherein the actuator is coupled to a system configured to apply a force to move the actuator, wherein the actuator is a piston.

6. (canceled)

7. The apparatus of claim 5, wherein the system comprises:
a biased spring coupled to the piston; and
an anchor coupled to the biased spring, wherein the anchor comprises a retaining pin, wherein removing the retaining pin from the anchor is configured to release the biased spring to actuate the piston in each chamber.

8. The apparatus of claim 1, further comprising a third chamber;
wherein each of the first chamber, the second chamber, and the third chamber comprise an outlet channel comprising a valve;
wherein each of the outlet channels converge to the mixing channel; and
wherein the valve is configured to allow fluids to flow from the first chamber, the second chamber, and the third chamber to the mixing channel.

9. (canceled)

10. (canceled)

11. (canceled)

12. The apparatus of claim 1, further comprising a separation unit between from the mixing channel and the delivery system, wherein the delivery system is a hypodermic needle, a cannula, a catheter, or a microneedle array.

13. (canceled)

14. A system for point-of-care administration, the system comprising:
a first chamber for receiving a first solution comprising nucleic acids in a first solvent;
a second chamber for receiving a second solution comprising lipids in a second solvent;
a mixing channel downstream the first chamber and the second chamber for receiving the first solution and the second solution, wherein the mixing channel is in fluid communication with the first chamber and the second chamber for mixing the first solution and the second solution to produce a third solution comprising lipid encapsulated nucleic acids; and
a delivery system downstream from the mixing channel, wherein the delivery system is in fluid communication with the mixing channel.

15. The system of claim 14, wherein the first solvent comprises a water-based solution or a pH-modified water-based solution, and wherein the second solvent comprises an alcohol.

16. (canceled)

17. (canceled)

18. (canceled)

19. The system of claim 14, wherein the second chamber comprises one or more lipids, sterols, and surfactants.

20. (canceled)

21. The system of claim 14, wherein each of the first chamber and the second chamber further comprise an actuator configured to force the first solution and the second solution to the mixing channel;
wherein the actuator is a piston;
wherein the piston is coupled to a biased spring; and
wherein the piston and biased spring are coupled to an anchor comprising a retaining pin, wherein removing the retaining pin is configured to release the biased spring to actuate the piston in each chamber.

22. (canceled)

23. (canceled)

24. (canceled)

25. The system of claim 14, further comprising a third chamber comprising an aqueous solution;
wherein each of the first chamber, the second chamber, and the third chamber include an outlet channel comprising a valve;
wherein each of the outlet channels converge to the mixing channel; and
wherein the valve is configured to allow fluids to flow from the first chamber, the second chamber, and the third chamber to the mixing channel.

26. (canceled)

27. (canceled)

28. (canceled)

29. The system of claim 14, further comprising a separation unit between from the mixing channel and the delivery system;
wherein the separation unit comprises a membrane;
wherein the separation unit is configured to receive a dialysis solution; and
wherein the separation unit is configured to flow the dialysis solution counter to the third solution.

30. (canceled)

31. (canceled)

32. (canceled)

33. A method for synthesizing and administering particle-based therapeutics, the method comprising:
providing an apparatus comprising:
a first chamber;
a second chamber proximal to the first chamber;
an actuator coupled to the first chamber and the second chamber;
a mixing channel downstream the first chamber and the second chamber, wherein the first chamber and the second chamber are each in fluid communication with the mixing channel; and
a delivery system downstream from the mixing channel, wherein the delivery system is in fluid communication with the mixing channel;
filling the first chamber with a first solution comprising nucleic acids in a first solvent;
filling the second chamber with a second solution comprising lipids in a second solvent;
applying a force to each of the first chamber and the second chamber to force the first solution and the second solution into the mixing channel;
forming a third solution comprising lipid encapsulated nucleic acids in the mixing channel; and
administering the lipid encapsulated nucleic acids through the delivery system.

34. The method of claim 33, wherein the step of forming and administering occur concurrently.

35. The method of claim 33, wherein the first solvent comprises a water-based solution and the second solvent comprises an alcohol.

36. (canceled)

37. (canceled)

38. The method of claim 33, wherein the actuator is a piston;
wherein the piston is coupled to a biased spring;
wherein the piston and biased spring are coupled to an anchor comprising a retaining pin; and
wherein the method further comprises removing the retaining pin to release the biased spring to actuate the piston in each chamber to force the first solution and the second solution to the mixing channel.

39. (canceled)

40. (canceled)

41. (canceled)

42. The method of claim 33, further comprising a third chamber comprising an aqueous solution;
wherein each of the first chamber, the second chamber, and the third chamber include an outlet channel comprising a valve; and
wherein each of the outlet channels converge to the mixing channel.

43. (canceled)

44. (canceled)

45. The method of claim 42, wherein the method further comprises actuating the valve to allow contents in the first chamber, the second chamber, and the third chamber to flow to the mixing channel.

46. The method of claim 33, wherein the apparatus further comprises a separation unit including a membrane, wherein the membrane separates the separation unit into a first region and a second region, wherein the method further comprises:
flowing the third solution in a first direction in the first region of the separation unit; and
flowing a dialysis solution in a second direction opposing the first direction in the second region of the separation unit,
wherein the dialysis solution removes components from the third solution.
